(19) United States
(12) Patent Application Publication
(10) Pub. No.: US 2019/0244680 A1
(43) Pub. Date: Aug. 8, 2019

(54) SYSTEMS AND METHODS FOR GENERATIVE MACHINE LEARNING

(71) Applicant: D-WAVE SYSTEMS INC., Burnaby (CA)

(72) Inventors: Jason Tyler Rolfe, Vancouver (CA); Karsten J. Kreis, Vancouver (CA)

(21) Appl. No.: 16/270,273

(22) Filed: Feb. 7, 2019

Related U.S. Application Data
(60) Provisional application No. 62/627,579, filed on Feb. 7, 2018.
(60) Provisional application No. 62/757,012, filed on Nov. 7, 2018.

(51) Int. Cl. G16B 40/30; G06N 7/00; G06N 20/00; G16H 50/20

(52) U.S. Cl. CPC G16B 40/30; G16H 50/20; G06N 20/00; G06N 7/005

(57) ABSTRACT

Machine-learning (ML) techniques and systems are advantageously employed in areas such as psychiatric genetics, and analysis of gene expression. Generative ML may be performed over large datasets to infer new pleiotropic effects of genetic variants in multiple neuropsychiatric diseases. Deep ML may infer information about psychiatric genetics. ML may realize an efficient estimation of multi-disease genetic and environmental correlation matrices. ML may obtaining protein expression data and generating a mapping between the protein expression data and at least one disease. ML may be performed on a data that that jointly models multiple diseases. Quantum processing can be advantageously employed in ML scenarios.

### FIELD

This disclosure generally relates to machine learning, and in particular to generative machine learning, computational approaches for psychiatric genetics, and analysis of gene expression.

### BRIEF SUMMARY

The present application describes systems and methods for generative machine learning over large datasets to infer new pleiotropic effects of genetic variants in multiple neuropsychiatric diseases. The systems and methods described in the present application can be advantageously implemented, at least in part, using a quantum computer.

The present application describes novel computational approaches for psychiatric genetics. These approaches can use deep learning and quantum computing. The systems and methods described in the present application can facilitate automatic extension of existing annotations of human genetic variants, and of protein expression markers in the brain, to numerous neuropsychiatric diseases by utilizing large medical and genetic datasets.

The approaches described in the present application can provide efficient estimation of multi-disease genetic and environmental correlation matrices using a generative machine learning method. The method can optionally be implemented, at least in part, using a quantum computer (also referred to in the present application as a quantum processor). The estimation can include higher-order disease correlations.

A benefit of the approaches described in the present application is that it becomes tractable to analyze a large health records dataset, for example, an electronic database containing data on more than 150 million people (also referred to in the present application as subjects) for multiple diseases (for example, 50 psychiatric diseases).

The systems and methods described in the present application include using protein expression data to generate a mapping between protein expression data and disease.

### BRIEF DESCRIPTION OF THE SEVERAL VIEWS OF THE DRAWING(S)

In the drawings, identical reference numbers identify similar elements or acts. The sizes and relative positions of elements in the drawings are not necessarily drawn to scale. For example, the shapes of various elements and angles are not necessarily drawn to scale, and some of these elements may be arbitrarily enlarged and positioned to improve drawing legibility. Further, the particular shapes of the elements as drawn, are not necessarily intended to convey any information regarding the actual shape of the particular elements, and may have been solely selected for ease of recognition in the drawings.

FIG. 1 is a schematic diagram of an example computing system including a digital computer and, optionally, an analog computer according to the present disclosure.

FIG. 2 shows an example implementation of a variational autoencoder (VAE) according to the present disclosure.

### DETAILED DESCRIPTION

### Introductory Generalities

In the following description, certain specific details are set forth in order to provide a thorough understanding of various disclosed implementations. However, one skilled in the relevant art will recognize that implementations may be practiced without one or more of these specific details, or with other methods, components, materials, etc. In other instances, well-known structures associated with computer systems, server computers, and/or communications networks have not been shown or described in detail to avoid unnecessarily obscuring descriptions of the implementations.

Unless the context requires otherwise, throughout the specification and claims that follow, the word “comprising” is synonymous with “including,” and is inclusive or open-ended (i.e., does not exclude additional, unrecited elements or method acts).

Reference throughout this specification to “one implementation” or “an implementation” means that a particular feature, structure or characteristic described in connection with the implementation is included in at least one implementation. Thus, the appearances of the phrases “in one implementation” or “in an implementation” in various places throughout this specification are not necessarily all referring to the same implementation. Furthermore, the particular features, structures, or characteristics may be combined in any suitable manner in one or more implementations.

As used in this specification and the appended claims, the singular forms “a,” “an,” and “the” include plural referents unless the context clearly dictates otherwise. It should also be noted that the term “or” is generally employed in its sense including “and/or” unless the context clearly dictates otherwise.

The terms “user”, “item”, and “rating” are used throughout this specification for convenience, as these terms are widely used in the art. Unless the context clearly dictates otherwise, such references refer generally to arbitrary dimensions in an input space (in the case of “user” and “item”) and to values lying in the input space (in the case of “rating”). Similarly, the terms “row”, “column”, and “vector” are used in the specification and claims to refer more generally to the dimensions of the input space, but these terms (as used herein) do not imply any specific directionality or data structure. For example, the use of “row” and “column” does not require or suggest that data is stored as a formal matrix in memory, and the use of “vector” does not require that a vector's constituent elements be stored contiguously in memory. To aid the reader, this specification will generally assume, without loss of generality, that users are elements along the row dimension and items are elements along the column dimension.

The headings and Abstract of the Disclosure provided herein are for convenience only and do not interpret the scope or meaning of the implementations.

### Computing Systems

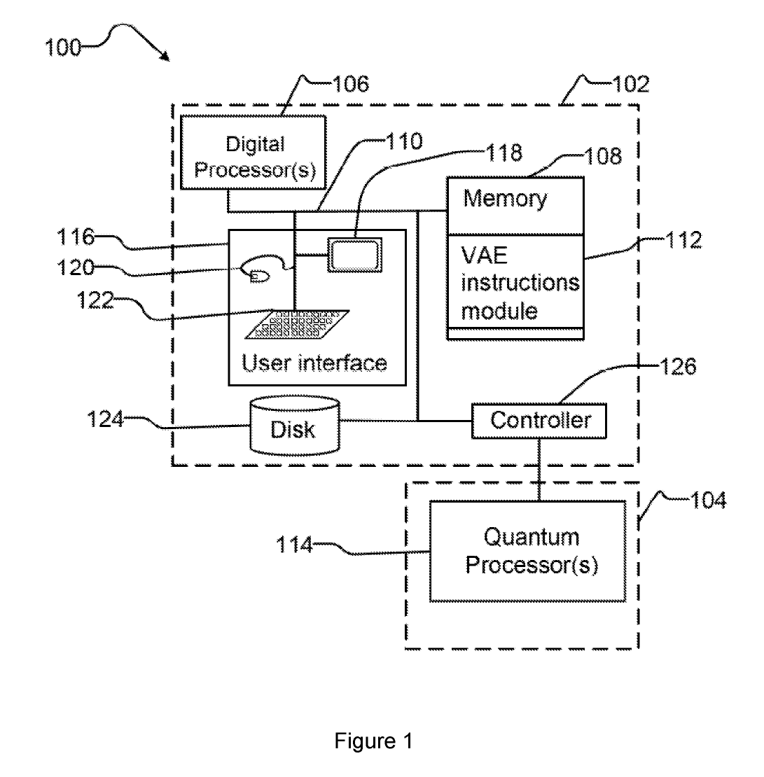

FIG. 1 illustrates a computing system 100 comprising a digital computer 102. The example digital computer 102 includes one or more digital processors 106 that may be used to perform classical digital processing tasks. Digital computer 102 may further include at least one system memory 108, and at least one system bus 110 that couples various system components, including system memory 108 to digital processor(s) 106. System memory 108 may store a VAE instructions module 112.

The digital processor(s) 106 may be any logic processing unit or circuitry (e.g., integrated circuits), such as one or more central processing units (“CPUs”), graphics processing units (“GPUs”), digital signal processors (“DSPs”), application-specific integrated circuits (“ASICs”), programmable gate arrays (“FPGAs”), programmable logic controllers (“PLCs”), etc., and/or combinations of the same.

In some implementations, computing system 100 comprises an analog computer 104, which may include one or more quantum processors 114. Digital computer 102 may communicate with analog computer 104 via, for instance, a controller 126. Certain computations may be performed by analog computer 104 at the instruction of digital computer 102, as described in greater detail herein.

Digital computer 102 may include a user input/output subsystem 116. In some implementations, the user input/output subsystem includes one or more user input/output components such as a display 118, mouse 120, and/or keyboard 122.

System bus 110 can employ any known bus structures or architectures, including a memory bus with a memory controller, a peripheral bus, and a local bus. System memory 108 may include non-volatile memory, such as read-only memory (“ROM”), static random access memory (“SRAM”), Flash NAND; and volatile memory such as random access memory (“RAM”) (not shown).

Digital computer 102 may also include other non-transitory computer- or processor-readable storage media or non-volatile memory 124. Non-volatile memory 124 may take a variety of forms, including: a hard disk drive for reading from and writing to a hard disk (e.g., magnetic disk), an optical disk drive for reading from and writing to removable optical disks, and/or a solid-state drive (SSD) for reading from and writing to solid-state media (e.g., NAND-based Flash memory). The optical disk can be a CD-ROM or DVD, while the magnetic disk can be a rigid spinning magnetic disk or a magnetic floppy disk or diskette. Non-volatile memory 124 may communicate with digital processor(s) via system bus 110 and may include appropriate interfaces or controllers 126 coupled to system bus 110. Non-volatile memory 124 may serve as long-term storage for processor- or computer-readable instructions, data structures, or other data (sometimes called program modules) for digital computer 102.

Although digital computer 102 has been described as employing hard disks, optical disks and/or solid-state storage media, those skilled in the relevant art will appreciate that other types of nontransitory and non-volatile computer-readable media may be employed, such magnetic cassettes, flash memory cards, Flash, ROMs, smart cards, etc. Those skilled in the relevant art will appreciate that some computer architectures employ nontransitory volatile memory and nontransitory non-volatile memory. For example, data in volatile memory can be cached to non-volatile memory. Or a solid-state disk that employs integrated circuits to provide non-volatile memory.

Various processor- or computer-readable instructions, data structures, or other data can be stored in system memory 108. For example, system memory 108 may store instruction for communicating with remote clients and scheduling use of resources including resources on the digital computer 102 and analog computer 104. Also, for example, system memory 108 may store at least one of processor executable instructions or data that, when executed by at least one processor, causes the at least one processor to execute the various algorithms described elsewhere herein, including machine learning related algorithms. For instance, system memory 108 may store a VAE instructions module 112 that includes processor- or computer-readable instructions to provide a variational autoencoder. Such provision may comprise training and/or performing inference with a variational autoencoder, e.g., as described in greater detail herein.

In some implementations system memory 108 may store processor- or computer-readable calculation instructions and/or data to perform pre-processing, co-processing, and post-processing to analog computer 104. System memory 108 may store a set of analog computer interface instructions to interact with analog computer 104. When executed, the stored instructions and/or data cause the system to operate as a special purpose machine.

Analog computer 104 may include at least one analog processor such as quantum processor 114. Analog computer 104 can be provided in an isolated environment, for example, in an isolated environment that shields the internal elements of the quantum computer from heat, magnetic field, and other external noise (not shown). The isolated environment may include a refrigerator, for instance a dilution refrigerator, operable to cryogenically cool the analog processor, for example to temperature below approximately 1° Kelvin.

Computing Genetic and Environmental Correlations Between Traits

Conventionally, genetic and environmental correlations between traits can be computed by using a multivariate, generalized linear mixed model with a probit link function. In statistics, a probit model is a type of regression where the dependent variable can take only two values. A link function provides a relationship between a linear predictor and the mean of a distribution function. The probability of an individual having a disease can be measured by an underlying Gaussian latent variable. The probability of an individual having a disease is also referred to in the present application as the liability. A conventional multivariate generalized linear mixed model can support the inference of a number of factors influencing disease liability and its variation. The factors can include genetic effects associated with pedigree, environmental effects shared by couples, environmental effects shared by siblings, environmental effects shared within families, and unique environmental effects of the subject.

The outcome vector in a linear regression y shows a case (y=1) or control (y=0) status for a disease on the observed scale. The liability [symbol] =Φ⁻¹(y), where Φ is the cumulative distribution function (CDF) of the standard normal distribution, and the liability can be expressed as follows:

[symbol]

=Xβ+a+c+s+ƒ+e

where X is a design matrix, β is a vector for fixed effects of age and gender, a is a vector of random, additive genetic effects based on pedigree, c is a vector of environmental effects shared by a couple, s is a vector of environmental effects shared by siblings, ƒ is a vector of environmental effects shared by a family, and e is a vector of unique effects of the subject. The design matrix is a matrix of values of explanatory variables of a set of subjects. Each row represents an individual subject, and successive columns correspond to variables and their specific values for that subject.

See, for example, Sorensen D. et al., “Likelihood, Bayesian, and MCMC methods in quantitative genetics”, Springer Science & Business Media (2007) for a derivation of a genetic correlation coefficient and an environmental correlation coefficient expressed in terms of variance components. Owing to the binary nature of phenotypic data, the variance components can be estimated using Bayesian methods, for example with a package suitable for fitting Bayesian mixed models such as MCMCglmm. The estimation can be computationally intensive, and can take millions of core-hours for even a moderate number of disease pairs.

Generalizing to Unobserved Latent Variables and Nonlinear Interactions Using Variational Autoencoders

One aspect of the systems and methods described in the present application is an extension of the generalized linear mixed model to a nonlinear hierarchical generative model. The nonlinear hierarchical generative model can be highly nonlinear. Another aspect of the systems and methods described in the present application is the joint modeling of multiple diseases in a dataset. Learning the parameters of a nonlinear hierarchical generative model would be impractical using conventional statistical techniques. The systems and methods described in the present application leverage the flexibility and efficiency of a variational autoencoder, and its hierarchical and discrete extensions, to make learning the parameters of the model achievable in practice.

Denoting the observed data by x and the latent variables by z, variational autoencoders are generative models p(x, z) paired with variational approximations to the posterior probability distribution of the latent variables q(z|x), trained by maximizing the evidence lower bound (ELBO) on the log-likelihood:

[symbol]

=log p(x)−KL[q(z|x)∥p(z|x)]

[symbol]

= [symbol] _{q(z|x)}[log p(x|z))]−KL[q(z|x)∥p(z)]

Conventionally, a generative model p(x, z) can be defined in terms of a) a prior probability distribution p(z) over latent variables z, and b) a conditional likelihood of the observed variables given the latent variables p(x|z). The prior probability distribution p(z) is also referred to in the present application as the prior. The approximating posterior probability distribution q(z|x) can be defined so that samples can be constructed using a deterministic function of the input x and an independent random variable. The approximating posterior probability distribution q(z|x) is also referred to in the present application as the approximating posterior. A re-parameterization technique can be used to backpropagate the gradient of the ELBO into q(z|x). Using the approach described here, the training process can be efficient.

Typically, a variational autoencoder can use a zero-mean, identity-covariance Gaussian prior distribution function p(z). A conditional likelihood p(x|z) can be selected to be a product of independent Gaussian distributions with means and variances defined by a neural network applied to a latent representation z. The present application describes systems and methods for improving the performance of the variational autoencoder by using a hierarchical approximating posterior and prior. In some implementations, the approximating posterior and/or the prior of a hierarchical variational autoencoder can be conditioned on one or more observed covariates, such as the age and sex of a subject. Conditioning can be analogous to a semi-supervised variational autoencoder.

A conditional likelihood probability distribution p(x|z) can be formed from a product of independent Bernoulli distributions over multiple diseases for a given subject, the probability distribution conditioned on latent variables that can, for example, include genetic and environmental, and latent variables that characterize the subject. A probability of each disease can be a respective output of a neural network applied to the subject's latent variables z. In some implementations, the neural network can be a linear transformation.

Latent variables can be helpful in capturing one or more hidden causes underlying the observed data. For example, latent variables can be helpful in capturing genetic and/or environmental contributions to disease. In some cases, hidden causes, and their associated latent variables, can be shared with other subjects such as members of a subject's family, and their differential contributions can be distinguished on that basis. There is some evidence, for example in pleiotropy and positive environmental correlations, that suggests a relatively small number of latent variables can explain much of the variance in health data. See, for example, Wang et al. “Classification of common human diseases derived from shared genetic and environmental determinants”, Nature Genetics, 49(9):1319, (2017). Pleiotropy occurs when one gene influences two or more seemingly unrelated phenotypic traits.

By analyzing multiple diseases experienced by a subject group (e.g., an individual subject's family) in a unified manner, the technology described in the present application can identify a cause that may be giving rise to multiple conditions across the subject group.

For example, a family can sometimes be represented by the mother's genetics, the father's genetics, the parents' shared environment, and the siblings' shared environment. Genetic information can be shared between children and their parents. Parents are generally more likely to be genetically independent. One approach is to assume an infinitesimal model of genetic linkage to disease, in which one or several quantitative traits can be described as the sum of a genetic and a non-genetic component, the first being distributed within families as a normal random variable centered at the average of the parental genetic components, and with a variance independent of the parental traits.

If there are multiple genes, each of which can make a small contribution to the risk of a disease, then a subject's genetic latent variable can be an average of the two parents', since the subject inherits half of their genes from each parent. Latent variables can also capture an unshared variation associated with each member of a family. Individual variation can be modeled with a hierarchy of latent variables, with some latent variables at a lower layer of the hierarchy than some genetic and environmental variables.

In the approximating posterior probability distribution, each subject and sibling of the subject can be processed separately (using shared parameters). Hidden representations of the subject and their siblings can subsequently be pooled using, for example, max-pooling and/or average pooling.

Genetic latent variables of both parents, as well as at least some environmental latent variables, can depend on the parents' data, and the data of the subject and their siblings. Other latent variables can be particular to the subject, and can be conditioned on applicable genetic and environmental variables, as well as on input from the subject.

Age, sex, and other observed covariates can be provided as arguments to p(x|z), and can be available to components of an approximating posterior q(z|x). As a consequence, variability explainable by observed covariates need not be attributed to latent variables.

Once a model has been trained, an average variance due to a selected latent variable can be evaluated when other latent variables are held constant. A correlation between diseases resulting from genetic and/or environmental factors can be evaluated similarly. In one approach, the methods described in the present application can provide an analysis of how genetic and environmental latent variables can combine to control at least some of the other elements of the hierarchical generative model. The sum of independent Gaussian latent variables can be Gaussian, and a vector of linear combinations of Gaussian random variables can be Gaussian with an analytically tractable covariance matrix.

The quality of a model relative to conventional approaches can be assessed using an k-fold cross-validation of the log-likelihood. In k-fold cross-validation, a sample is randomly partitioned into k equally sized subsamples. Of the k subsamples, one subsample is retained as validation data for testing the model. The remaining (k−1) subsamples can be used as training data. The cross-validation process can be repeated k times, with each of the k subsamples used once as validation data. The k results can then be averaged to provide an estimation of the log-likelihood.

Inferring Common Disease Etiology Via Independent Component Analysis

Independent component analysis (ICA) can linearly transform a multidimensional input such that the resulting signals vary independently. Mutual information between signals can be defined such that mutual information between independent signals is zero. One canonical approach for finding independent components is to minimize, or at least reduce, their mutual information. In the context of health data, each such independent component can correspond to a weighted group of diseases that are strongly correlated, and independent of other weighted groups of disease, and thus may be associated with a distinct cause.

One approach to ICA includes maximizing, or at least increasing, the entropy of a selected nonlinear transformation of the independent components. Under some circumstances, this approach can correspond to maximizing, or at least increasing, the mutual information between an input and the extracted components. Moreover, this approach can be at least approximately equivalent to training a generative model as described above, and with a non-Gaussian prior probability distribution that is rotationally asymmetric. Though variational autoencoders conventionally use Gaussian priors, they are compatible with other choices of prior probability distribution. For example, some implementations of sparse coding use a Laplace prior probability distribution.

A variational autoencoder using a non-Gaussian prior probability distribution can be applied to independent component analysis of MRI images, for example. When used in conjunction with a linear decoder, clusters of independent diseases can be read from model parameters.

A generative model can retain its expressive power provided the approximating posterior and prior are hierarchical. Latent variables in a hierarchical generative model are not independent, whether they have rotational symmetry, or not. One approach is to restrict attention to the top-most latent layer of the hierarchy, for which the latent variables can be independent of each other (though not necessarily independent of the succeeding layers). Another approach is to relax the assumption that causes of disease are independent. In practice, it is reasonable to expect a degree of correlation between genes and environmental properties. Hierarchy can also render a generative model nonlinear. The mean of layer n can be the output of a neural network operating on layers 1 through (n−1).

A variational autoencoder can be used to perform independent component analysis. In one implementation, a variational autoencoder can be used to perform independent component analysis by increasing a scaling on the KL term in the expression for the evidence lower bound (ELBO) on the log-likelihood. In one implementation, the variational autoencoder includes a regularization term of the form β·KL[q(z|x)∥p(z)] where β is a scaling factor. The ELBO on the log-likelihood can be expressed as follows:

[symbol]

= [symbol] _{q(z|x)}[log p(x|z)]−β·KL[q(z|x)∥p(z)]

The mutual information I(x, z) can be expressed as follows:

I(x,z)= [symbol] ₓ[KL[p(z|x)∥p(z)]]

Since q(z|x)≈p(z|x), the ELBO on the log-likelihood can be approximated as follows:

[symbol]

=log p(x)−KL[q(z|x)∥p(z|x)]−(β−1)·I(x,z)

The variational encoder, in this case, can be at least closely related to minimizing, or at least reducing, the mutual information between observed and latent variables.

Integration of Proteomic Data

Proteomics is the large-scale study of proteomes. A proteome is a set of proteins produced in an organism, system, or biological context. A set of diseases diagnosed in a subject can characterize their state of health. A latent representation learned by a variational autoencoder to explain the subject's state of health can summarize their physiological state.

An advantage of a variational autoencoder is that it can accommodate diverse forms of data. Health data for a subject can be augmented, for example, with proteomic data such as expression levels of a collection of diagnostic proteins. Latent variables, including shared genetic and environmental variables characterizing a subject's family, can learn to explain a health phenotype expressed in terms of disease susceptibility and/or protein expression.

A product of Bernoulli distributions can parameterize a conditional likelihood probability distribution p(x|z) for a disease state. A product of independent Gaussian distributions on the log-expression levels can parameterize the conditional likelihood of protein expression levels. Over-dispersion and under-dispersion relative to a Gaussian distribution, and correlations between the proteins, can be captured by latent variables.

A relationship between disease susceptibility and protein expression can be inferred using a joint variational autoencoder, whether proteomic data is available for the same population as the disease state, or not. The present application describes systems and methods to use a shared generative model p(z)·p(x|z) for training distinct approximating posteriors q(z|x) on proteomic and health information datasets. While there is no guarantee that individual latent variables will capture the same underlying physiological properties in the two approximating posteriors, they will necessarily use the same prior probability distribution and projection to the observed variables, and consequently can be expected to learn to have at least similar statistics. Coupling between the two approximating posteriors can be made more reliable if there is a subset of families of subjects for which both proteomic and health data are available.

Capturing Clustered Structure with Quantum-Ready Discrete Variational Autoencoders

Continuous latent variables of traditional variational autoencoders can typically capture smooth distributions over observed variables. A shortcoming of variational autoencoders defined over continuous latent variables is that they can tend to force clusters together in latent space, whether the observed data is clustered, or not. The result may be inconsistent with underlying health data, for example if there are distinct subpopulations with different health risks, such as may arise if subjects can be clustered by race, socioeconomic status, and/or place of residence.

In contrast, discrete variational autoencoders can directly capture the clustered nature of the data via a set of binary latent variables over which a restricted Boltzmann machine prior can be defined.

To train a discrete variational autoencoder, samples can be drawn from the restricted Boltzmann machine prior over the binary latent variables. Sampling (and even approximate sampling) from a restricted Boltzmann machine cannot be performed in polynomial time. Conventional approaches typically use approximate sampling algorithms for which performance degrades as training progresses, thereby potentially adversely affecting the results.

The distributions being sampled can be very similar to those from which a quantum annealer (such as the D-Wave quantum computer) natively samples. A quantum annealer can be used to provide more efficient and effective training of the models described above.

Analyzing Gene Expression Data with Variational Autoencoders

The transcriptome includes the set of RNA molecules in a cell or a population of cells. The transcriptome may refer to RNA or messenger RNA (mRNA), depending on context. The transcriptome is also described in the present application as including a rate of RNA expression of genes in the genome. The transcriptome can provide an incisive and accessible characterization of the state of a tissue.

RNA can be transcribed into proteins. Proteins can constitute a primary building block of a cell, as well as be a substrate for controlling biochemistry. In some cases, proteins can directly mediate a process in the cell. In other cases, proteins can regulate a process in the cell. Some RNA transcripts can modulate the rate of expression of other proteins. Even when RNA transcription levels do not directly mediate a cellular process, they may be modulated by the process, and so they may be diagnostic even when they are not causal.

Proteins can be synthesized and regulated depending upon the functional need in the cell. The blueprints for proteins are stored in DNA and decoded by highly regulated transcriptional processes to produce messenger RNA (mRNA). The rate at which gene are transcribed from DNA to RNA can be efficiently measured using RNA sequencing and microarrays, for example. The message coded by an mRNA can be translated into a protein. Transcription is the transfer of information from DNA to mRNA, and translation is the synthesis of protein based on a sequence specified by mRNA.

The transcriptome can be used to analyze genes that are differentially expressed based upon the phenotype of the organism. For instance, individuals with (partially) heritable diseases express certain genes at a higher or lower rate than typical individuals. These differentially expressed genes may suggest targets for pharmacological intervention, facilitating the creation of novel drugs. Modules of genes with correlated expression levels across tissues or phenotypic variations can be identified, and used to infer functional relationships. In combination with DNA sequencing, the transcriptome can also be used to identify the interaction between genotype and gene expression. Gene expression is a process by which information from a gene is used in the synthesis of a functional gene product. These products are often proteins.

RNA and DNA sequencing can produce data with high dimensionality. Obtaining samples in large numbers can be challenging and expensive. As a result, the number of genes measured is often orders of magnitude greater than the number of samples. For instance, available gene expression datasets for schizophrenia and autism spectrum disorder measure the activity of at least 2×10⁴ genes, and only contain approximately 5×10² samples.

Given such unbalanced datasets, traditional frequentist statistical techniques can require onerous amounts of regularization. A statistical test performed independently on each gene can need to be corrected for multiple comparisons (e.g., via Bonferroni correction or Benjamini-Hochberg false discovery rate), allowing only stronger interactions to be detected if there are more genes than samples.

Even a linear regression from the data to a target value of interest can fail if the number of measurements is greater than the number of samples. Moreover, traditional transcriptome analyses are based on linear models of observed covariates, whereas the molecular dynamics that underlie gene regulation are highly nonlinear. Negative feedback loops that can induce nonlinear saturation effects are pervasive in cellular dynamics. Proteins and other cellular components can interact with each other, and with the environment, and the results of these interactions can affect transcription rates. Unmeasured properties of the subject, and processing procedure can constitute latent variables that can affect a measured gene transcription rate. The unmeasured properties are typically not represented in the models that underlie conventional systems and methods. The resulting higher-order, nonlinear statistics can manifest in a linear model without hidden latent variables as unexplained variance, squandering the already limited statistical power of small transcriptome datasets.

Unmodeled, higher-order correlations can have a pernicious interaction with small gene expression datasets. More samples can be needed to accurately estimate the variance of a random variable than to accurately estimate the mean because the more extreme samples tend to drive the estimated variance. Such extreme samples are less common in the Poisson distribution and its relatives that can be used to model the structure of gene reads. The correlates of differential gene expression in the genotype or phenotype can be more readily identified if the covariate-driven change is large compared to the measured variance in each condition. Small datasets tend to increase the uncertainty of the measured variance in each condition, thereby imposing the use of more conservative statistical tests to further inflate the estimate of the variance, and reducing the ability to detect more modest differences between conditions.

Differential Gene Expression

Determining if a distribution of gene reads differs significantly between experimental conditions can depend on an accurate estimate of the variance. Conventional approaches to analyzing differential gene expression typically reduce the number of samples required to estimate the variance by assuming that the variance is a function of the mean, and/or by taking similar values across multiple genes (embodied in a shared prior over all genes).

There can be an ambiguity between the expression rate of genes and a measurement rate of expressed genes. There are methods that can estimate the library depth with a median-of-ratios method, for example. Other methods can use a weighted trimmed mean of the log expression ratios (trimmed mean of M values also referred to in the present application as TMM).

One existing approach uses a linear model on the log-read count, rather than a generalized linear model, and so assumes the log-transformed data is normally distributed. This approach can be effective in practice for log-intensities of microarray data. The approach can be augmented by modeling the standard deviation of the log-read count as a function of the mean.

Another existing approach assumes a negative binomial model, with the mean expression rate per condition and gene scaled by a regularized library depth, and with the variance perturbed from the mean by a function of the transcription rate (scaled by the regularized library depth squared). Differential expression can be evaluated by computing the probability of the observed counts in different conditions (for a single gene). A shortcoming of the approach is that it does not use an explicit generalized linear model.

Yet another existing approach assumes a negative binomial model, where the mean of the gene expression rate is the exponential of a linear function of the covariates, and the variance is a quadratic function of the mean. A likelihood ratio test can be used to assess whether individual coefficients are different from zero. The generalized linear model of this approach induces a negative binomial distribution, with a different parameterization and statistical test than other approaches.

Denoising

In traditional approaches to denoising, the noise is assumed to induce an additive perturbation on the true gene expression data, and noise sources are assumed to be independent and combine additively. Hidden sources of noise can be modeled as random variables dependent on known noise sources. A shortcoming is that Gaussian noise models may not be appropriate for count data.

Surrogate variable analysis (SVA) first computes the principal components of the residual expression data, after accounting for the observed covariates (equivalent to the right singular vectors), selects those that explain more variance than expected on unstructured (permuted) data, identifies the genes that are significantly correlated with each significant principal component, re-computes the principal components on residual data restricted to the identified significant genes, selects the principal component on the reduced residual matrix most similar to the original principal component, and then fits a joint linear model where the surrogate variables have the selected vectors. The extracted surrogate variables can be included as explicit covariates in subsequent analyses. Their values can be deterministically computed from the observed data, and can serve as the basis for additional linear modeling in conventional differential expression analyses.

To the extent that the surrogate variables capture the variation in the data, the per-gene variance of the model used to analyze differential gene expression can remain small, and the analysis can be sensitive to smaller differences in the mean. The surrogate variables can be continuous and adjustable, in contrast to the fixed, discrete observed covariates, and so can more effectively represent the observed gene expression data.

It has been observed that even simple sources of variation can be highly nonlinear. For example, there are instances where log-transformed RPKM (reads per kilobase million) RNA sequencing (RNA-Seq) expressions can segregate into three prominent clusters that can be correlated with sequencing depth. A shortcoming of existing methods is that Gaussian latent variables with linear conditional likelihoods can be unable to capture this structure.

Module Extraction

Weighted gene co-expression network analysis (WGCNA) is an existing approach that can be used to produce a set of modules, where elements of a module can exhibit correlated variation across samples of a dataset. WGCNA can include a) computing a correlation matrix of gene expression vectors, b) applying an element-wise nonlinearity, c) applying a transformation with a heuristic measure of topological overlap, and d) performing an average linkage hierarchical clustering.

In another conventional approach, the algorithm for the reconstruction of accurate cellular networks (ARACNE) constructs an undirected graph over the genes, where edges indicate the presence of strong regulatory interactions that cannot be explained by other multi-step paths in the network. ARACNE can include a) approximating the observed distribution of RNA expression levels with a mixture Gaussians, one at each data point, b) computing the mutual information between pairs of genes, and c) removing edges for which the mutual information is below a threshold, or for which there exists a length-2 pathway where both mutual information values are larger than that of a direct connection. The resulting sparse graph is generally expected to contain edges corresponding to direct transcriptional interactions.

A shortcoming of WGCNA can be that it is linear while molecular dynamics that underlie gene regulation are highly nonlinear. A shortcoming of ARACNE can be that it assumes regulatory interactions only occur between pairs of genes. In practice, genes do not only interact via direct transcriptional regulation of one gene by another protein. Protein and other cellular components can interact with each other, and with the environment, and the results of these interactions can have an impact on transcription rates.

In summary, it has been observed that, in actual gene expression dynamics, there are latent variables beyond the observed gene transcription rates, and interactions are high-order and nonlinear.

Nonlinear Approaches

Variational autoencoders can be applied to gene expression data. See for example Wray G. et al, “Evaluating deep variational autoencoders trained on pan-cancer gene expression”, arXiv:1711.04828 (2017), which used a variational autoencoder to pre-train features that could be used by a linear classifier to predict cancer type from tumor gene expression data.

Variational Autoencoders Applied to Gene Expression Data

One aspect of the systems and methods described in the present application is an extension of the generalized linear mixed model to a nonlinear hierarchical generative model. The nonlinear hierarchical generative model can be highly nonlinear. The systems and methods described in the present application leverage the flexibility and efficiency of a variational autoencoder, and its hierarchical and discrete extensions, to provide effective systems and methods suitable for analyzing gene expression and sequencing data.

Denoting the observed data by x and the latent variables by z, variational autoencoders are generative models p(x, z) paired with variational approximations to the posterior probability distribution of the latent variables q(z|x), trained by maximizing the evidence lower bound (ELBO) on the log-likelihood:

[symbol]

=log p(x)−KL[q(z|x)∥p(z|x)]

[symbol]

= [symbol] _{q(z|x)}[log p(x|z)]−KL[q(z|x)∥p(z)]

Conventionally, a generative model p(x, z) can be defined in terms of a) a prior probability distribution p(z) over latent variables z, and b) a conditional likelihood of the observed variables given the latent variables p(x|z). The prior probability distribution p(z) is also referred to in the present application as the prior. The approximating posterior probability distribution q(z|x) can be defined so that samples can be constructed using a deterministic function of the input x and an independent random variable. The approximating posterior probability distribution q(z|x) is also referred to in the present application as the approximating posterior. A re-parameterization technique can be used to backpropagate the gradient of the ELBO into q(z|x). Using the approach described here, the training process can be efficient.

Typically, a variational autoencoder can use a zero-mean, identity-covariance Gaussian prior distribution function p(z). A conditional likelihood p(x|z) can be selected to be a product of independent Gaussian distributions with means and variances defined by a neural network applied to a latent representation z. The present application describes systems and methods for improving the performance of the variational autoencoder by using a hierarchical approximating posterior and prior. In some implementations, the approximating posterior and/or the prior of a hierarchical variational autoencoder can be conditioned on one or more observed covariates, such as the age and sex of a subject, sequencing depth, guanine-cytosine (GC) bias, gene-length, RNA quality measures, library type, read depth, postmortem interval, and/or brain tissue pH. Conditioning can be analogous to a semi-supervised variational autoencoder. Latent variables can capture unobservable technical factors, such as how well the technique of polymerase chain reaction (PCR) amplification worked, and how much ribosomal-RNA depletion occurs during library preparation stages.

A Poisson distribution can be used to model a case where genes are read at random from a pool of genetic material. A limitation can be that the variance of a Poisson distribution is equal to the mean, whereas, in practice, gene reads can exhibit overdispersion, i.e., the variance can be greater than the mean under a selected experimental condition.

Existing approaches such as those described above can use a negative binomial distribution instead of a Poisson distribution. A negative binomial distribution can capture over-dispersion in the data. A negative binomial distribution can be equivalent to a mixture of Poisson distributions with a gamma prior on the Poisson rate parameter.

The systems and methods described in the present application can include modeling gene reads by a Poisson distribution conditioned on latent variables, to accommodate multiple sources of variation. Over-dispersion can be handled by a distribution over the Poisson rate parameter. For example, a negative binomial distribution assumes a gamma distribution over the Poisson rate parameter, and a gamma prior can be used in a variational autoencoder to realize a negative binomial marginal distribution over the read counts. While the choice of the gamma prior in the negative binomial distribution can be computationally convenient, its inclusion in the variational autoencoder can be optional.

In one implementation, log λ of the Poisson distribution is modeled directly. Having no non-negativity constraint, it can capture relevant changes in gene expression as proportional, rather than additive. The effect of library depth on gene reads can also be rendered additive rather than multiplicative.

Regularization of the dispersion between genes can be mediated by the prior over the Poisson rate parameter in the variational autoencoder. The prior on the Poisson rate parameter can include a shared prior distribution across genes, even though the Poisson rate must be independently sampled in the posterior for each element of the dataset, rather than shared across instances of each gene.

The per-gene parameters can be the real parameters, and the shared prior can behave as a regularization term (also referred to in the present application as a regularizer). This approach can provide a consistent method for training a top-level prior over the genes, as well as the per-gene parameters.

The resulting hierarchical variational autoencoder can capture nonlinear relationships between covariates and gene expression. For instance, a set of covariates may each increase expression of a group of genes in isolation, and yet their marginal effects may saturate when they are present simultaneously. Similarly, a pair of covariates may both need to be present for either one of them to affect gene expression. Some covariates may additively increase the number of gene reads, whereas others may multiplicatively scale the number of reads. While these relationships can be incompatible with traditional linear models, they can be captured by a hierarchical variational autoencoder.

In accordance with the systems and methods described in the present application, a hierarchical variational autoencoder can be used to model the effect of observed covariates, accommodate unobserved sources of variance, and decompose variability in gene expression into independent modules in a single unified framework.

A quantity of biological interest (and which is typically Poisson distributed) is the gene transcription rate, which can be scaled by a measurement rate to obtain the number of reads. The percentage of reads associated with a gene is typically not modeled because the percentage of each gene can be affected by the transcription rate of other genes. While the approach could explicitly model the measurement rate, another option is to estimate it using a trimmed mean of M values (TMM) method, for example.

The transcriptome is typically evaluated using large numbers of cells, and so variation can be due to genetic, environmental, and technical differences. Since there are significant differences in gene expression between different cell types, some of the variation can be due to the ratio of different cell types in the sample, in which case the cell types may be found to define prominent latent axes of variation.

Differential Gene Expression

One use of the technology described in the present application is to test whether expression of a single gene can behave differently depending on disease condition. The expression of the gene may not be consistently greater in one disease condition than another. There may be other latent variables (either observed or hidden) on which the effect of disease condition on gene expression may depend. In some cases, the effect of disease condition on gene expression may even change sign depending on other latent variables.

In conventional approaches, surrogate variables typically compete with observed covariates to explain gene expression data. An advantage of using a variational autoencoder as described in the present application is that the latent variables of a variational autoencoder can be a mechanism through which observed covariates can explain the data. The observed covariates can shape the distribution in a low-dimensional latent space from which the observed variables are generated.

Since a latent variable can serve at least a similar function as an observed covariate, it is preferable not to use a likelihood ratio test to compare a model with and without an observed covariate. A test can be performed to determine whether the correct value of the covariate better explains the held-out test data (i.e., test data not used in fitting the model) than an incorrect value of the covariate, or whether the trained model has learned different distributions for each value of the observed covariate.

Bayes Factor Due to Changing Disease Condition

In accordance with the systems and methods described in the present application, one approach is to do the following: a) train a generative model for which a disease condition is an observed latent variable using a training subset of the data, b) for elements in a mutually exclusive test set, infer the distribution over the selected gene, conditioned on the expression of other genes, and c) fix the disease condition to possible values in turn. This can be accomplished, for example, by evaluating the probability of a set of genes, and the set of genes except for the selected one, given the disease condition, as follows:

$$p ( {gene}_{i} \vert {gene}_{j \neq i} , disease ) = \frac{p ( gene \vert disease )}{p ( {gene}_{j \neq i} \vert disease )}$$

Other observed covariates, including technical covariates, can be held constant. In one implementation, the technology evaluates a Bayes factor of the held-out gene expression value for the correct versus incorrect disease condition using an importance-weighted estimate of the log-likelihood, for example.

Since the log likelihoods are evaluated on a test dataset distinct from that used to train the parameters of the model, the true disease condition will only induce a better fit if there is a difference between expression of the gene depending upon the disease condition. In contrast, if the disease condition is independent of the held-out gene, then the predicted distribution of the held-out gene, and the log-likelihood of its true value, should be the same for all disease conditions. Since models corresponding to different values of the disease condition have the same number of parameters, the Akaike information criterion (an estimator of the relative quality of statistical models for a given data set) is equivalent to the log-likelihood.

Bayes factors corresponding to independent samples can be multiplied, so that even relatively weak effects of disease on gene expression can compound (in some cases, exponentially) across the test dataset. If there is no differential gene expression, the log of the Bayes factor can be proportional to the square root of the test dataset size, and the per-data-set-element variance can be small. When there are many possible disease conditions, the estimate can be conservative.

If the remaining (non-held-out) genes are strongly indicative of a disease condition, forcibly changing it may throw the network into an untrained state, in which no predictions are accurate. In another implementation, an approach is to restrict the analysis to elements of the test set for which values of the disease condition induce samples from the approximating posterior for which the prior probability is above a predetermined threshold. These gene expression configurations can be ambiguous, and can be plausibly associated with various disease conditions.

In one implementation, an importance-weighted approximation to the log-likelihood, that more accurately samples from the true posterior distribution, can be used in place of the ELBO. Even if a disease condition is incompatible with a gene expression configuration, the conditional likelihood p(x|z) can dominate the prior p(z) in the true posterior p(z|x) provided there are many more genes than latent variables.

In one implementation, to evaluate the log-likelihood of the gene expression configuration conditioned on counterfactual disease conditions, a new approximating posterior can be trained in the absence of information regarding the disease condition. The approach uses the disease-condition-agnostic approximating posterior to evaluate the importance-weighted log-likelihood estimate for at least some disease conditions. The approach can be expected to yield plausible configurations of latent variables, even if a gene expression configuration is improbable under a specific disease condition.

Test Marginal Generative Distribution of Selected Gene Conditioned on Disease Condition

In another implementation, the approach includes training the model, then ignoring the training data and randomly sampling from the trained model with the disease condition fixed at values in turn, and evaluating whether the disease condition affects the gene in question. The (hidden) latent variables can have potentially different meanings depending on the disease condition, so the latent variables are not held constant. Instead, the observed covariates (e.g., age, technical factors) are held constant and a Komolgorov-Smirnov test used to check whether expression of the selected gene differs between disease conditions for a specific fixed set of observed covariates, integrating out unobserved latent variables by sampling. This tests differential expression, and can identify changes in the distribution even when the mean remains constant. The test is insensitive to differential correlation between genes.

Rather than perform the equivalent test on a limited set of raw data, one approach is as follows: a) first build a model of the data, and then b) draw a suitable number of samples from it. For a single learned model, changing a latent variable such as the disease condition can have some effect on the distribution of genes. One solution is to train multiple such models, and pool their samples to sample effectively from the posterior distribution over the model parameters given the training data. If there is no consistent relationship between the disease condition and the expression of a selected gene in the data, then, when averaged across multiple trained models, there is expected to be little or no difference in the generated distributions conditioned on the disease state.

In one implementation, tests can be combined across different values of the observed covariates, for example by independently retraining the model for each value of the observed covariates on which differential expression is tested.

If the disease condition is independent of the expression of a selected gene, then changing the expression of the gene in question should not alter the posterior distribution of the disease condition, which is only determined by the other genes. In contrast, changing the selected gene should change the overall log-likelihood of the data. A test can be performed to determine whether the gradient of the posterior distribution of the disease condition, with respect to the selected gene, is different from zero. In one implementation, the approach includes additional regularization of the generative model. More efficient use of limited data can be made by evaluating p(gene expression)|disease condition), rather than p(disease condition|gene expression).

Semi-Supervised Classification of Disease Condition

In addition to identifying genes and modules for which expression is affected by the disease condition, the systems and methods described in the present application can predict the disease condition from gene expression data. In one implementation, a semi-supervised variational autoencoder is trained to model p(transcriptome) and p(disease|transcriptome) and the results assessed using k-fold cross validation (described elsewhere in the present application). The approach using a variational autoencoder described in the present application can yield improved risk scoring from gene expression data over existing approaches such as using elastic nets to predict disease state from gene expression data.

This approach can also be used to identify differential gene expression. In contrast to the previous generative model p(disease)·p(latent|disease), the semi-supervised approach uses the following generative model p(latent)·p(disease|latent). The latent representation can be used to reduce the dimensionality of the input used to predict the disease condition, thereby regularizing the prediction.

Expression Quantitative Trait Loci

Genotype information can influence transcriptome prediction in at least a similar manner as observed technical covariates. The approximating posterior and/or the prior can be conditioned on the genotype. In the case of the approximating posterior, prediction of the disease condition can yield a polygenic risk assessment. In the case of the prior, an assessment of differential gene expression as a function of a genotype variable can yield expression quantitative trait loci (also referred to in the present application as an eQTL). A quantitative trait locus (QTL) is a section of DNA (the locus) which correlates with variation in a phenotype (the quantitative trait). Expression quantitative trait loci (eQTLs) are genomic loci that contribute to variation in expression levels of mRNAs (messenger RNAs).

The massive number of genotype variables makes the identification of spurious eQTLs, likely, and these can mask true eQTLs in the process. This can be mitigated, at least in part, by projecting a large number of SNP/CNV (single nucleotide polymorphisms/copy number variation) variables through a smaller number of latent variables, which then generate a larger number of gene expression predictions. Geometric prior information about the location of SNPs/CNVs can be incorporated in their projection to the latent variables; nearby mutations should project to the same sparse set of latent variables. This may be discovered naturally when the genotype information is forced through a low-dimensional bottleneck, with single latent variables representing contiguous regions of the genome.

Traditional linear approaches can require that an independent coupling be learned between each mutation and the expression of genes. In contrast, the observed gene expression configurations are, at least usually, expected to be close to a low-dimensional sub-manifold of the space of possible gene expressions. It can be more efficient to infer the effect of mutations on the low-dimensional manifold, since it can be characterized by fewer parameters.

In another implementation, conditioning the model of gene expression on genotype can be replaced by conditioning a model of genotype (and gene expression) on gene expression. The encoder sees gene expression, and the decoder predicts gene expression and genotype. If the model is conditioned on disease label, it can be determined whether the predicted distribution for a genotype variable changes significantly when the disease label is altered. This can be done using an at least similar Bayes factor approach as described for analyzing differential gene expression.

In another implementation, an underpowered distribution of the genotype conditioned on the latent space of gene expressions can be constructed. The average gradient of the predicted genotype variables conditioned on each of the gene expression or latent variables can be computed. Significant deviations from uncorrelated genes can be evaluated using a perturbation test on the genotype variables (for example by randomly permuting the values for a selected genotype variable amongst elements of the training set, and evaluating the distribution of gradients). Since there are usually more genotype variables than gene expression variables, the contribution of the genotype to the log-likelihood can be suitably down-weighted.

Given p(transcriptome|disease) and p(genome|transcriptome), it is possible to derive p(disease|genome). The transcriptome, and the latent variables that represent it, can be a suitable latent space for dissecting nonlinear interactions in the genome. Groups of interacting mutations can mediate disease. If a group of mutations mediating a disease is reflected in the details of the transcriptome, then a generative model of the genome conditioned on the transcriptome can be used to discover and represent the clusters.

Inferring Gene Clusters Via Independent Component Analysis

Independent component analysis (ICA) can be used to linearly transform a multidimensional input such that the resulting signals vary independently. Each such independent component can correspond to a group of inputs that are strongly correlated and independent of other such groups, and thus constitute a gene activity cluster in the context of gene expression data. Mutual information can be defined such that the mutual information between independent signals is zero. One approach to finding independent, or at least partially independent, components is to minimize, or at least reduce, their mutual information. Minimizing, or at least reducing, mutual information between components is related to minimizing, or at least reducing, the joint entropy of the extracted independent components relative to that of a corresponding Gaussian distribution.

Another approach to inferring gene clusters via ICA is to maximize, or at least increase, entropy of a selected nonlinear transformation of the independent components, which under certain circumstances corresponds to maximizing, or at least increasing, the mutual information between the input and the extracted component. This can be at least approximately equivalent to training a generative model with a non-Gaussian prior that is rotationally asymmetric.

Though variational autoencoders conventionally use Gaussian priors, they are compatible with other choices of prior probability distribution. For example, some implementations of sparse coding use a Laplace prior probability distribution.

A variational autoencoder using a non-Gaussian prior probability distribution can be applied to independent component analysis of MRI images, for example. When used in conjunction with a linear decoder, clusters of independent diseases can be read from model parameters.

A generative model can retain its expressive power provided the approximating posterior and prior are hierarchical. Latent variables in a hierarchical generative model are not independent, whether they have rotational symmetry, or not. One approach is to restrict attention to the top-most latent layer of the hierarchy, for which the latent variables can be independent of each other (though not necessarily independent of the succeeding layers). Another approach is to relax the assumption that causes of disease are independent. In practice, it is reasonable to expect a degree of correlation between genes and environmental properties. Hierarchy can also render a generative model nonlinear. The mean of layer n can be the output of a neural network operating on layers 1 through (n−1).

A variational autoencoder can be used to perform independent component analysis. In one implementation, a variational autoencoder can be used to perform independent component analysis by increasing a scaling on the KL term in the expression for the evidence lower bound (ELBO) on the log-likelihood. In one implementation, the variational autoencoder includes a regularization term of the form β·KL[q (z|x)∥p(z)] where β is a scaling factor. The ELBO on the log-likelihood can be expressed as follows:

[symbol]

= [symbol] _{q(z|x)}[log p(x|z)]−β·KL[q(z|x)∥p(z)]

The mutual information I(x, z) can be expressed as follows:

I(x,z)= [symbol] ₓ[KL[p(z|x)∥p(z)]]

Since q(z|x)≈p(z|x), the ELBO on the log-likelihood can be approximated as follows:

[symbol]

=log p(x)−KL[q(z|x)∥p(z|x)]−(β−1)·I(x,z)

The variational encoder, in this case, can be at least closely related to minimizing, or at least reducing, the mutual information between observed and latent variables.

While some latent variables may correspond to gene modules, others may capture unobserved sources of technical variation. To distinguish differentially expressed gene modules from condition-independent sources of technical variation, the prior distribution conditioned on the possible disease states can be compared using a Kolmogorov-Smirnov test. Given enough samples, a statistically significant difference can generally be found. An option is to test whether the approximating posterior distribution of a latent variable is different, when marginalized over elements of the dataset with a single value of the disease condition, from distributions for other values of the disease condition.

Individual Targeting of Chemotherapeutic Agents

The prognosis and preferred treatment regime for cancer can be strongly predicted by the genetics of the tumor cells. The genetics of the tumor is typically reflected in its transcriptome. Since cancerous cells may contain many novel mutations, the transcriptome may be easier to analyze. Genetic repair mechanisms are often disabled in cancerous cells, leading to more mutations than in healthy cells.

Every distinct mutation may have unique effects on the biology of the cancer. To predict treatment efficacy from tumor genetics, the effect of various mutations can need to be inferred or learned. Mutations can have their effects through modification of RNA transcription rates, changes in the translation of RNA into proteins, and/or alteration of the function of the final proteins. Variations in translation and function often have further effects on the translation rate of other genes. Since the relative concentration of mRNA (and, by extension, proteins) can characterize a metabolic state of a cell, it can serve as an effective probe of the effect of mutations. While the dimensionality of the space of possible mutations is linear in the total number of base pairs in the genome, the space of gene expression patterns has dimensionality equal to the number of genes, and is typically at least an order of magnitude smaller.

The space of realizable gene expression patterns can be a low dimensional sub-manifold within the space of possible configurations, and can be learned, at least in part, from measurements of healthy cells. There is typically less statistical structure in the space of mutations.

Cancer is not one disease, even when restricted to a single organ. Each tumor can depend on a subset of oncogenic mutations, each of which can make specific perturbations to various biochemical pathways. Chemotherapeutic drugs typically attack certain selected pathways, and can be most effective when their method of action is appropriately aligned with the unique biochemistry of a tumor. Since the transcriptome can characterize the biochemical nature of a cancer, it provides an opportunity to predict the efficacy of chemotherapeutic drugs from a suitable characterization of the transcriptome of a tumor. Drugs predicted to shrink a tumor most effectively can be chosen over drugs less likely to be as effective.

The systems and methods described in the present application can be used to select amongst commercially available chemotherapeutic drugs, and to facilitate development and FDA approval of new, less toxic, targeted chemotherapeutic drugs. Many potential chemotherapy drugs act via circumscribed biochemical pathways, and so may not be effective against tumors selected based on organ of origin, and histological properties. Different tumors can perturb different biochemical pathways, and so, in test, a drug may appear to have little or no impact on some tumors including those that do not rely on pathway targeted by the drug under test. As a result, targeted therapies may fail an FDA approval process that has no mechanism for selecting an appropriate pool of test subjects (patients). A more effective approach to classifying tumors (such as the approach described in the present application) may help to facilitate FDA approval, and later prescription, of these drugs.

Drug Discovery

The systems and methods described in the present application can be used to find new drugs, for example. In one implementation, the approach includes a) separately modeling i) changes in the transcriptome associated with a disease, and ii) changes in the transcriptome induced by drugs, and b) selecting drugs (or combinations of drugs) that can induce transcriptome changes opposite to those associated with the disease. Since drugs act on the environment, rather than on the genome, it is possible they are reflected in the transcriptome by secondary and higher-order effects.

Another approach is to use the systems and methods described in the present application to infer changes in the proteome induced by the transcriptome, and match drugs to induced changes in proteome associated with the disease.

Genes identified as being differentially expressed in tumors might correspond to proteins that could be targeted by novel cancer drugs.

In addition to the structure amongst the gene expression vectors, there can also be exploitable relationships amongst the cancer drugs. Groups of drugs or other therapeutics typically target common biochemical pathways, and so are generally expected to have correlated effects on cell lines. The relationship between gene expression and cancer drugs can be complex, and, in at least some cases, nonlinear.

One approach is to combine gene expression and drug or therapeutics information at the last linear layer of a classifier. The systems and methods described in the present application can merge a hidden vector capturing gene expression (e.g., the approximating posterior of a variational autoencoder, or the output of a neural network taking the approximating posterior as input) with a parameter vector associated with each drug (either by element-wise multiplication or concatenation). The joint representation can be passed to a neural network to predict the effect of a drug or other therapeutic on the cell line. The approach includes a nonlinear generalization of the trace-norm regularization of drug characterization vectors.

Inferring Non-Linear Genetic Effects and Environmental Interactions

Some implementations of the presently-disclosed VAEs may be adapted to estimate heritability and/or correlations between environmental and genetic conditions for one or more diseases. For example, a VAE may receive information on families and/or individuals (e.g. clinical health data, family relationships, and/or genomic data) and decompose it in the latent space into a lower-dimensional representation.

In some implementations, this lower-dimensional representation is explicitly based on the biology of the subjects. For example, the latent space may comprise a plurality of latent subspaces, with each subspace comprising one or more latent variables. The decoder, which generates representations of families and/or individuals from representations in the latent space, processes different subspaces differently. For instance, one or more subspaces may be directed to genetic information and one or more other subspaces may be directed to non-genetic information (e.g. environmental information).

For example, a subspace that is directed to encode genetic data may be processed by the decoder to generate representations based on a covariance matrix corresponding to the expected genetic covariance between related or unrelated persons. Non-genetic subspaces (e.g. a family environmental subspace, a parents' environmental subspace, a siblings' environmental subspace, and/or one or more unique-effects subspaces) may possess different covariance characteristics, e.g. such that a parents' environmental subspace contributes only (or disproportionately more strongly) to the construction of a representation of parents and not (or less strongly) to children.

One potential advantage of this approach is that it is not necessarily limited to analyzing diseases independently or in pairs, a common limitation of past techniques. A VAE may receive as input to the encoder, for example, clinical information on individuals (e.g. organized into families) which describes several diseases. One example training dataset used by the inventors represents, for each individual, their diagnoses (or lack thereof) for nearly 200 diseases, along with metadata such as the individual's sex, age, and/or familial relationships. The VAE can ingest, at the encoder, each of those nearly 200 disease datapoints for each individual, along with the associated metadata. This allows for the VAE to jointly learn and model the various diseases represented in the training set. In some implementations, the encoder and/or decoder are conditioned on observed covariates of disease, such as age and/or sex. Some training datasets also, or alternatively (to familial data), provide explicit genetic data for individuals; this may include, for example, SNP sequences. This can also be provided to the encoder as input.

In some implementations, the VAE receives a family as each input sample at the encoder and generates a family for output at the decoder, thereby allowing familial relationships to be provided implicitly and allowing for joint training and generation of related individuals. (Some implementations ensure uniformly-sized input data by requiring a fixed number of parents and/or children as input and allowing some to be flagged as dummies, with dummy entries being ignored during encoding and decoding.)

Consider an example VAE which represents families having two parents and two children, with each of the parents being biologically related to each of the children but not to each other. (Other family structures may be represented; this example is provided for convenience, not as a limitation.) The genetic relationships between these individuals can be represented by a covariance matrix M:

$$M = [ \begin{matrix}1 & 0 & 1 / 2 & 1 / 2 \\ 0 & 1 & 1 / 2 & 1 / 2 \\ 1 / 2 & 1 / 2 & 1 & 1 / 2 \\ 1 / 2 & 1 / 2 & 1 / 2 & 1\end{matrix} ]$$

Where (for the purposes of this example) the first row corresponds to the mother, the second row corresponds to the father, the third row corresponds to the first child, and the fourth row corresponds to the second child. Generating the members of a family in the decoder may involve, for at least the genetic latent subspace(s), sampling from a multivariate distribution (e.g. a multivariate Gaussian distribution with covariance matrix M), sampling from i.i.d. distributions for each family member and inducing correlations explicitly (e.g. by determining parents' samples independently and subsequently determining children's samples based on a mixture of the parents' samples and a random noise component), and/or otherwise determining appropriately-correlated samples. For instance, a genetic latent variable c for a child may be constructed as follows:

$$c = \frac{1}{2} m + \frac{1}{2} f + \frac{1}{\sqrt{2}} n$$

where m is the corresponding latent variable for the mother, ƒ is the corresponding latent variable for the father, and n is a noise component (e.g. corresponding to Mendelian genetic randomization).

Some implementations use Gaussian distributions for sampling from genetic latent variables to correspond to the observed distribution of genetic traits (which is typically Gaussian), although other distributions may be used (e.g. Bernoulli distributions in implementations with discrete latent variables).

Other, non-genetic subspaces' latent variables may be sampled from differently by the decoder. For example, latent variables in a family environmental subspace may be used by the decoder when generating all family members, whereas latent variables in a parents' environmental subspace may be used only when generating parents. For example, that input to the decoder may be “turned off” (e.g. reduced to 0 or otherwise ignored) when generating portions of the output relating to the children. Similarly, a children's environmental subspace may be used only when generating children. In some implementations, rather than turning off such inputs (e.g. children's latent space variables) when generating non-corresponding family members (e.g. parents), the weight attached to them is reduced relative to corresponding family members (e.g. children).

Architecting the model in this way is expected to cause the VAE to tend to represent genetic causes of disease largely in the genetic latent space(s) and environmental causes of disease largely in environmental latent space(s). The parsimonious behavior of VAE models is expected to tend to avoid representing non-genetic causes in genetic latent variables and vice-versa, through the mechanism of explaining-away. That is, since genetic latent variables (for instance) provide correlations which fit genetic causes well but tend to fit environmental causes poorly, VAEs will tend to shift the representational weight of those causes to the genetic causes.

The foregoing VAE, once trained, has a variety of potential applications. For example, the decoder (i.e. the generative model) may be used to estimate the heritability of diseases, interactions between genetic and environmental factors, and genetic features such as dominance and epistasis. For instance, to estimate heritability, one may generate a set of latent representations where the genetic latent subspace(s) of the latent representations are fixed to a particular state (which we will call the genetic latent state) and the non-genetic latent subspace(s) are allowed to vary between representations. The decoder may then be used to generate a corresponding set of families (and/or individuals, depending on the implementation) based on the latent representations, from which an expected value of the resulting families may be determined for that genetic latent state. By repeating this for multiple genetic latent states, one can determine the variance of the output over the genetic states (e.g. by determining the variance of the expected values.) Similarly, by unlocking all genetic and environmental latent variables, one may determine the variance of the overall model over the latent space. The ratio of the genetic variance to the overall variance for a given disease provides an estimator of the heritability of that disease.

Another example application relates to predicting the probability of a diagnosis for any of the modelled diseases for a given individual. For example, one may pass an individual's information through the encoder to produce a latent representation of the individual and then, in the decoder, generate predictions based on the latent representation (provided as input to the decoder). This prediction may comprise predictions for each of the modelled diseases or a subset thereof. For the aforementioned example training dataset, an individual could receive predictions of susceptibility to hundreds of diseases at once.

Such predictions may be conditioned on age. For example, where the decoder is conditioned on age, the person's age may also be passed as a parameter to the decoder; the result is that the output of the decoder will tend to predict diagnoses for the individual at that age (e.g. the individual's current age or a future age). Medical practitioners may use such information to order tests (e.g. for high-probability diseases, which may have gone undiagnosed previously) and/or to recommend preventative measures to reduce the risk of contracting diseases for which an individual is at risk in the future. The encoder may also, optionally, be conditioned on the individual's age. By advancing the age on which the conditional likelihood is conditioned, the model may identify diseases which increase in probability for a particular individual as they age, allowing the likely long-term development of the individual's health state to be mapped out in advance.

Such prediction of future diseases may be refined, in suitable circumstances, by providing the VAE (and particularly the approximating posterior) on only a subset of diseases diagnosed in a family/individual before a fixed date during training, but requiring the conditional likelihood to predict all diseases, including those diagnosed after the cutoff imposed on the input to the approximating posterior (e.g. by incorporating later-dated diseases into the loss term). The resulting approximating posterior will tend to aim to infer latent representations which explicitly predict into the future.

In some implementations, the VAE may provide discrete latent variables in addition to (or instead of) continuous latent variables. This may provide additional representative power in the cases of certain high-penetrance, single-gene diseases such as (for example) Huntington's chorea. As described in greater detail elsewhere herein, such discrete latent variables may be provided by a discrete VAE and may involve sampling from a quantum processor and/or representation of all or a portion of the latent space (e.g. genetic subspace(s)) via a restricted Boltzmann machine.

In an example implementation, the population means for all diseases x̅ may be calculated from the trained model based on:

x̅=∫ƒ_{θ}(z_{G},z_{E},zₑ,μ)p(z_{G})p(z_{E})p(zₑ)p(μ)dx_{*}dμ

where the terms are latent variables (e.g. Gaussian variables) corresponding to genetic causes (z_{G}), environmental causes (z_{E}), and unique effects (zₑ; i.e. effects which are specific to an individual); ƒ_{θ} is a neural network with parameters θ that maps latent variables to Bernoulli probabilities of the modelled diseases; μ represents the fixed effects (e.g. age, sex); and dz_{*} denotes integration over all latent variables z_{G}, z_{E}, zₑ. The term ƒ_{θ} may predict the mean of the conditional likelihood p(x|z), which may follow a Bernoulli distribution. The p(z_{*}) distributions are prior distributions over the relevant latent subspaces and p(μ) is a distribution over the fixed effects. Where the underlying dataset is representative of the population of interest, p(μ) may be estimated by sampling from the dataset rather than explicitly modelling the distribution.

The full expected-scale variance may be determined based on:

V_{P,exp}=∫(ƒ_{θ}(z_{G},z_{E},zₑ,μ)−x̅)²p(z_{G})p(z_{E})p(zₑ)p(μ)dzdμ

and the full observed-scale or data-scale variance may be determined based on:

V_{P,obs}=V_{P,exp}+∫ƒ_{θ}(z_{G},z_{E},zₑ,μ)(1−ƒ_{θ}(z_{G},z_{E},zₑ,μ))p(z_{G})p(z_{E})p(zₑ)p(μ)dzdμ

where the term ƒ_{θ}(z_{G}, z_{E}, zₑ, μ)(1−ƒ_{θ}(z_{G}, z_{E}, zₑ, μ)) denotes the variance of the Bernoulli distribution.

The expected phenotype of an individual with fixed genetic latent value z̅_̅{̅G̅}̅ may be determined based on:

x̅_{z}_{_{G}}=∫ƒ_{θ}(z_{G},z_{E},zₑ,μ)p(z_{E})p(zₑ)p(μ)dz_{E}dzₑdμ

and the total genetic variance on both the expected and observed data scales may be determined based on:

V_{z}_{_{G}}=∫(x̅z_{G}−x̅)²p(z_{G})dz_{G}

While the integral expressions above are not necessarily analytically solvable, they may be estimated efficiently by drawing samples from the generative model after training.

Using these variances (and/or estimates thereof), one may calculate heritabilities on the observed and expected scales based on:

$${H}_{obs}^{2} = \frac{{V}_{{\overline{x}}_{G}}}{{V}_{P , obs}} {H}_{exp}^{2} = \frac{{V}_{{\overline{x}}_{G}}}{{V}_{P , exp}}$$

These heritabilities can be considered broad-sense heritabilities because the model being used is not a linear and additive model (like the general linear mixed models sometimes used for estimating heritabilities). Instead, the VAE learns to represent a non-linear mapping from latent causes to disease diagnoses which is capable of representing dominance, epistasis, genetic-environmental interactions, and/or other causes which are not readily linearly representable.

Further statistical genetics quantities of interest, such as environmental effects and environmental and genetic correlation between diseases, may also be calculated from the trained model and may also provide broad-sense estimates.

The foregoing refers to individuals, fathers, mothers, and children for convenience, but the presently-disclosed systems and methods may be applied to non-human organisms as well. For example, the present disclosure may be applied to plants to facilitate plant breeding by isolating genetic causes of particular phenotypes or to predict which offspring (from the space of all possible offspring produced by pairs of plants in the dataset) would produce desired phenotypes across environments and/or in a given environment.

Extending to Multiple VAEs

Although the foregoing description refers generally to systems providing one VAE, the present disclosure is not limited to such implementations. Indeed, the presently-described systems and methods can, in certain circumstances, provide further advantages with the addition of one or more further VAEs.

Hierarchical and/or Discrete Variational Autoencoders

In some implementations, one or more VAEs use hierarchical prior and approximating posterior distributions (as described, for example, by Rolfe, “Discrete Variational Autoencoders”, arXiv:1609.02200v2 [stat.ML] 22 Apr. 2017, incorporated herein by reference). The approximating posterior and prior distributions may have matched structures. Such VAEs can, in certain circumstances, provide highly multi-modal distributions.

In some implementations, the one or more hierarchical VAEs provided by the system comprise discrete variational autoencoders (e.g., as described in the aforementioned paper). Such discrete VAEs may provide greater modelling power when representing multimodal distributions. For example, when a hierarchical approximating posterior is used, the DVAE can produce highly multi-modal approximating posteriors.

Collaborative Filtering

In some implementations, one or more VAEs are structured to represent the problem of predicting diseases via a collaborative filtering approach, e.g. as described in US Patent Application Nos. 62/598,880 and 62/658,461 and/or PCT Application No. US2018/065286. For instance, individuals may be represented as users and diseases as items, with a user-item matrix providing predictions for each individual-disease pairing. This enables joint modeling of diseases, which can be advantageous where (as is often the case) each individual has been tested for some diseases but not for others (or even for most diseases), leaving the model with an incomplete dataset to train over. Jointly modeling these individual-disease interactions allows the model to generate predictions for one disease based on the (other) diseases that have been diagnosed for this and/or other individuals.

Quantum Machine Learning

In some implementations the latent space of an encoder comprises a restricted Boltzmann machine (RBM). The system may comprise an interface with a quantum processor 114 (e.g., a quantum annealing processor). Training the VAE(s) may comprise computing a gradient of a KL term with respect to the RBM by representing a distribution by the quantum processor 114, such as a Boltzmann distribution and/or a quantum Boltzmann distribution, and executing the quantum processor to determine a sample output. Training the VAE(s) may further comprise obtaining the sample based on the sample output of the quantum processor. This may involve, for example, post-processing the sample output (e.g., to incorporate quantum error correction or otherwise process the sample output).

Variational Auto-Encoder

Unsupervised learning of probabilistic models is a technique for machine learning. Unsupervised learning of probabilistic models can facilitate tasks such as denoising to extract a signal from a mixture of signal and noise, and inpainting to reconstruct lost or corrupted parts of an image. Unsupervised learning of probabilistic models can also regularize supervised tasks such as classification.

One approach to unsupervised learning can include attempting to maximize the log-likelihood of an observed dataset under a probabilistic model. Equivalently, unsupervised learning can include attempting to minimize the KL-divergence from the data distribution to that of the model. While the exact gradient of the log-likelihood function is frequently intractable, stochastic approximations can be computed, provided samples can be drawn from the probabilistic model and its posterior distribution given the observed data.

The efficiency of using stochastic approximations to arrive at a maximum of the log-likelihood function can be limited by the poor availability of desirable distributions for which the requisite sampling operations are computationally efficient. Hence, applicability of the techniques can be similarly limited.

Although sampling can be efficient in undirected graphical models provided there are no loops present among the connections, the range of representable relationships can be limited. Boltzmann machines (including restricted Boltzmann machines) can generate approximate samples using generally costly and inexact Markov Chain Monte Carlo (MCMC) techniques.

Sampling can be efficient in directed graphical models comprising a directed acyclic graph since sampling can be performed by an ancestral pass. Even so, it can be inefficient to compute the posterior distributions over the hidden causes of observed data in such models, and samples from the posterior distributions are required to compute the gradient of the log-likelihood function.

Another approach to unsupervised learning is to optimize a lower bound on the log-likelihood function. This approach can be more computationally efficient. An example of a lower bound is the evidence lower bound (ELBO) which differs from the true log-likelihood by the KL-divergence between an approximating posterior distribution, q(z|x, ∅), and the true posterior distribution, p(z|x, θ). The approximating posterior distribution can be designed to be computationally tractable even though the true posterior distribution is not computationally tractable. The ELBO can be expressed as follows:

$$ℒ ( x , θ , φ ) = log p ( x \vert θ ) - KL [ q ( z \vert x , θ ) ∥ p ( z \vert x , θ ) ] = {\int }_{z} q ( z \vert x , φ ) log [ \frac{p ( x , z \vert θ )}{q ( z \vert x , φ )} ]$$

where x denotes the observed random variables, z the latent random variables, θ the parameters of the generative model and ϕ the parameters of the approximating posterior.

Successive optimization of the ELBO with respect to ϕ and θ is analogous to variational expectation-maximization (EM). It is generally possible to construct a stochastic approximation to gradient descent on the ELBO that only requires exact, computationally tractable samples. A drawback of this approach is that it can lead to high variance in the gradient estimate, and can result in slow training and poor performance.

The variational auto-encoder can regroup the ELBO as:

[symbol]

(x,θ,ϕ)=−KL[q(z|x,ϕ)∥p(z|θ)]+ [symbol] _{q}[log p(x|z,θ)].

The KL-divergence between the approximating posterior and the true prior is analytically simple and computationally efficient for commonly chosen distributions, such as Gaussians.

A low-variance stochastic approximation to the gradient of the auto-encoding term [symbol] _{q} can be backpropagated efficiently, so long as samples from the approximating posterior q(z|x) can be drawn using a differentiable, deterministic function ƒ(x, ϕ, ρ) of the combination of the inputs x, the parameters ϕ, and a set of input- and parameter-independent random variables ρ˜D. For instance, given a Gaussian distribution with mean m(x, ϕ) and variance v(x, ϕ) determined by the input, [symbol] (m(x, ϕ), v(x, ϕ)), samples can be drawn using

$$f ( x , φ , ρ ) = m ( x , φ ) + \sqrt{v ( x , φ )} \cdot ρ , where p ∼ ( 0 , 1 ) . \\ When such an f ( x , φ , ρ ) exists , \\ {𝔼}_{q ( z \vert x , ∅ )} [ log p ( x \vert z , θ ) = {𝔼}_{ρ} [ log p ( x \vert f ( x , ρ , ∅ ) , θ ) ] \\ \frac{\partial }{\partial φ} {𝔼}_{q ( z \vert x , ∅ )} [ log p ( x \vert z , θ ) ] = {𝔼}_{ρ} [ \frac{\partial }{\partial φ} log p ( x \vert f ( x , ρ , φ ) , θ ) ] \approx \frac{1}{N} {\sum }_{ρ ∼ D} \frac{\partial }{\partial ∅} log p ( x \vert f ( x , ρ , ∅ ) , θ ) , \qquad ( 1 )$$

and the stochastic approximation to the derivative in equation 1 is analytically tractable so long as p(x|z, θ) and ƒ(x, ρ, ∅) are defined so as to have tractable derivatives.

This approach is possible whenever the approximating posteriors for each hidden variable, qᵢ(zᵢ|x, ϕ), are independent given x and ϕ; the cumulative distribution function (CDF) of each qᵢ is invertible; and the inverse CDF each qᵢ, is differentiable. Specifically, choose [symbol] to be the uniform distribution between 0 and 1, and ƒᵢ to be the inverse CDF of qᵢ.

The conditional marginal cumulative distribution (CDF) is defined by

Fᵢ(x)=∫ₓ_{ᵢ}_{=-∞}^{x}p(xᵢ′|x₁, . . . ,xᵢ₋₁)

Since the approximating posterior distribution q(z|x, ϕ) maps each input to a distribution over the latent space, it is called the “encoder”. Correspondingly, since the conditional likelihood distribution p(x|z, θ) maps each configuration of the latent variables to a distribution over the input space, it is called the “decoder”.

Unfortunately, a multivariate CDF is generally not invertible. One way to deal with this is to define a set of CDFs as follows:

Fᵢ(x)=∫ₓ_{ᵢ}_{′=-∞}^{x}p(xᵢ′|x₁, . . . ,xᵢ₋₁)

and invert each conditional CDF in turn. The CDF Fᵢ(x) is the CDF of x₁ conditioned on all xⱼ where j<i, and marginalized over all xₖ where i<k. Such inverses generally exist provided the conditional-marginal probabilities are everywhere non-zero.

### Discrete Variational Auto-Encoders

The approach can run into challenges with discrete distributions, such as, for example, Restricted Boltzmann Machines (RBMs). An approximating posterior that only assigns non-zero probability to a discrete domain corresponds to a CDF that is piecewise-constant. That is, the range of the CDF is a proper subset of the interval [0, 1]. The domain of the inverse CDF is thus also a proper subset of the interval [0, 1] and its derivative is generally not defined.

The difficulty can remain even if a quantile function as follows is used:

Fₚ⁻¹(ρ)=inf{z∈ [symbol] :∫_{z′=-∞}^{z}p(z′)≥ρ}

The derivative of the quantile function is either zero or infinite for a discrete distribution.

One method for discrete distributions is to use a reinforcement learning method such as REINFORCE (Williams, http://www-anw.cs.umass.edu/˜barto/courses/cs687/williams92simple.pdf). The REINFORCE method adjust weights following receipt of a reinforcement value by an amount proportional to the difference between a reinforcement baseline and the reinforcement value. Rather than differentiating the conditional log-likelihood directly in REINFORCE, the gradient of the log of the conditional likelihood distribution is estimated, in effect, by a finite difference approximation. The conditional log-likelihood log p(x|z, θ) is evaluated at many different points z˜q(z|x, ϕ), and the gradient

$$\frac{\partial }{\partial φ} log q ( ( z \vert x , φ )$$

weighted more strongly when p(x|z, θ) differs more greatly from the baseline.

One disadvantage is that the change of p(x|z, θ) in a given direction can only affect the REINFORCE gradient estimate if a sample is taken with a component in the same direction. In a D-dimensional latent space, at least D samples are required to capture the variation of the conditional distribution p(x|z, θ) in all directions. Since the latent representation can typically consist of hundreds of variables, the REINFORCE gradient estimate can be much less efficient than one that makes more direct use of the gradient of the conditional distribution p(x|z, θ).

A discrete variational auto-encoder (DVAE) is a hierarchical probabilistic model consisting of an RBM, followed by multiple layers of continuous latent variables, allowing the binary variables to be marginalized out, and the gradient to backpropagate smoothly through the auto-encoding component of the ELBO.

The generative model is redefined so that the conditional distribution of the observed variables given the latent variables only depends on the new continuous latent space.

A discrete distribution is thereby transformed into a mixture distribution over this new continuous latent space. This does not alter the fundamental form of the model, nor the KL-divergence term of the ELBO; rather it adds a stochastic component to the approximating posterior and the prior.

One interpretation of the way that VAEs work is that they break the encoder distribution into “packets” of probability, each packet having infinitesimal but equal probability mass. Within each packet, the values of the latent variables are approximately constant. The packets correspond to a region in the latent space, and the expectation value is taken over the packets. There are generally more packets in regions of high probability, so more probable values are more likely to be selected.

As the parameters of the encoder are changed, the location of each packet can move, while its probability mass stays constant. So long as F_{q(z|x, ϕ)}⁻¹ exists and is differentiable, a small change in ϕ will correspond to a small change in the location of each packet. This allows the use of the gradient of the decoder to estimate the change in the loss function, since the gradient of the decoder captures the effect of small changes in the location of a selected packet in the latent space.

In contrast, REINFORCE works by breaking the latent representation into segments of infinitesimal but equal volume, within which the latent variables are also approximately constant, while the probability mass varies between segments. Once a segment is selected in the latent space, its location is independent of the parameters of the encoder. As a result, the contribution of the selected location to the loss function is not dependent on the gradient of the decoder. On the other hand, the probability mass assigned to the region in the latent space around the selected location is relevant.

Though VAEs can make use of gradient information from the decoder, the gradient estimate is generally only low-variance provided the motion of most probability packets has a similar effect on the loss function. This is likely to be the case when the packets are tightly clustered (e.g., if the encoder produces a Gaussian distribution with low variance) or if the movements of well-separated packets have a similar effect on the loss function (e.g., if the decoder is roughly linear).

One difficulty is that VAEs cannot generally be used directly with discrete latent representations because changing the parameters of a discrete encoder moves probability mass between the allowed discrete values, and the allowed discrete values are generally far apart. As the encoder parameters change, a selected packet either remains in place or jumps more than an infinitesimal distance to an allowed discrete value. Consequently, small changes to the parameters of the encoder do not affect most of the probability packets. Even when a packet jumps between discrete values of the latent representation, the gradient of the decoder generally cannot be used to estimate the change in loss function accurately, because the gradient generally captures only the effects of very small movements of the probability packet.

Therefore, to use discrete latent representations in the VAE framework, the method described herein for unsupervised learning transforms the distributions to a continuous latent space within which the probability packets move smoothly. The encoder q(z|x, ∅) and prior distribution p(z|θ) are extended by a transformation to a continuous, auxiliary latent representation ζ, and the decoder is correspondingly transformed to be a function of the continuous representation. By extending the encoder and the prior distribution in the same way, the remaining KL-divergence (referred to above) is unaffected.

In the transformation, one approach maps each point in the discrete latent space to a non-zero probability over the entire auxiliary continuous space. In so doing, if the probability at a point in the discrete latent space increases from zero to a non-zero value, a probability packet does not have to jump a large distance to cover the resulting region in the auxiliary continuous space. Moreover, it ensures that the CDFs Fᵢ(x) are strictly increasing as a function of their main argument, and thus are invertible.

The method described herein for unsupervised learning smooths the conditional-marginal CDF Fᵢ(x) of an approximating posterior distribution, and renders the distribution invertible, and its inverse differentiable, by augmenting the latent discrete representation with a set of continuous random variables. The generative model is redefined so that the conditional distribution of the observed variables given the latent variables only depends on the new continuous latent space.

The discrete distribution is thereby transformed into a mixture distribution over the continuous latent space, each value of each discrete random variable associated with a distinct mixture component on the continuous expansion. This does not alter the fundamental form of the model, nor the KL-divergence term of the ELBO; rather it adds a stochastic component to the approximating posterior and the prior.

The method augments the latent representation with continuous random variables ζ, conditioned on z, as follows:

q(ζ,z|x,ϕ)=r(ζ|x)·q(z|x,ϕ)

where the support of r(ζ|x) for all values of z is connected, so the marginal distribution q(ζ|x, ϕ)=Σ_{z}r(ζ|z)·q(z|x, ϕ) has a constant, connected support so long as 0<q(z|x, ∅)<1. The approximating posterior r(ζ|x) is continuous and differentiable except at the end points of its support so that the inverse conditional-marginal CDF is differentiable.

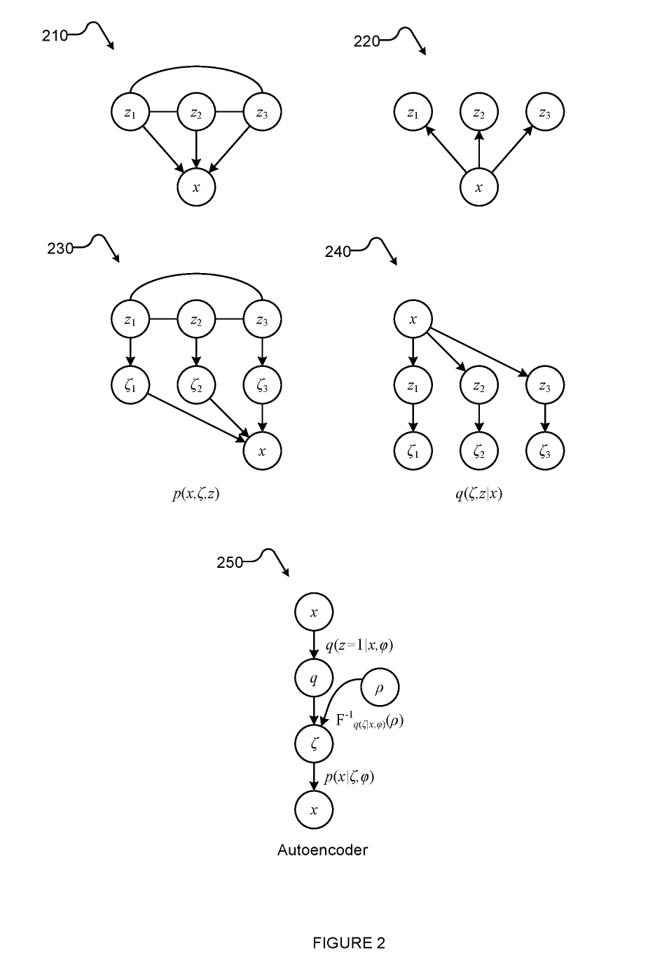

FIG. 2 shows an example implementation of a VAE. The variable z is a latent variable. The variable x is a visible variable (for example, pixels in an image data set). The variable ζ is a continuous variable conditioned on a discrete z as described above in the present disclosure. The variable ζ can serve to smooth out the discrete random variables in the auto-encoder term. As described above, the variable ζ generally does not directly affect the KL-divergence between the approximating posterior and the true prior.

In the example, the variables z₁, z₂, and z₃ are disjoint subsets of qubits in the quantum processor. The computational system samples from the RBM using the quantum processor. The computational system generates the hierarchical approximating posteriors using a digital (classical) computer. The computational system uses priors 210 and 230, and hierarchical approximating posteriors 220 and 240.

For the prior 230 and the approximating posterior 240, the systems adds continuous variables ζ₁, ζ₂, ζ₃ below the latent variables z₁, z₂, z₃.

FIG. 2 also shows the auto-encoding loop 250 of the VAE. Initially, input x is passed into a deterministic feedforward network q(z=1|x, ∅), for which the final non-linearity is the logistic function. Its output q, along with independent random variable ρ, is passed into the deterministic function F_{q(ζ|x, ∅)}⁻¹ to produce a sample of ζ. This ζ, along with the original input x, is finally passed to log p(x|ζ,θ). The expectation of this log probability with respect to ρ is the auto-encoding term of the VAE. This auto-encoder, conditioned on the input and the independent ρ, is deterministic and differentiable, so backpropagation can be used to produce a low-variance, computationally efficient approximation to the gradient.

The distribution remains continuous as q(z|x, ϕ) changes. The distribution is also everywhere non-zero in the approach that maps each point in the discrete latent space to a non-zero probability over the entire auxiliary continuous space. Correspondingly, p(ζ,z|θ) is defined as p(ζ, z|θ)=r(ζ|z)·p(z|θ), where r(ζ|z) is the same as for the approximating posterior, and p(x|ζ,z,θ)=p(x|ζ,θ). This transformation renders the model a continuous distribution over z.

The method described herein can generate low-variance stochastic approximations to the gradient. The KL-divergence between the approximating posterior and the true prior distribution is unaffected by the introduction of auxiliary continuous latent variables, provided the same expansion is used for both.

The auto-encoder portion of the loss function is evaluated in the space of continuous random variables, and the KL-divergence portion of the loss function is evaluated in the discrete space.

The KL-divergence portion of the loss function is as follows:

$$- KL [ q ( z \vert x , φ ) ∥ p ( z \vert θ ) ] = {\sum }_{z} q ( z \vert x , φ ) \cdot [ log p ( z \vert θ ) - log q ( z \vert x , φ ) ]$$

The gradient of the KL-divergence portion of the loss function in the above equation with respect to θ can be estimated stochastically using samples from the true prior distribution p(z|θ). The gradient of the KL-divergence portion of the lost function can be expressed as follows:

$$\frac{\partial KL ( q ∥ p )}{\partial θ} = - {〈 \frac{\partial {E}_{p} ( z \vert θ )}{\partial θ} 〉}_{q ( z \vert x , φ )} + {〈 \frac{\partial {E}_{p} ( z \vert θ )}{\partial θ} 〉}_{p ( z \vert θ )}$$

In one approach, the method computes the gradients of the KL-divergence portion of the loss function analytically, for example by first directly parameterizing a factorial q(z|x, ϕ) with a deep network g(x):

$$q ( z \vert x , φ ) = \frac{{e}^{- {E}_{q} ( z \vert x , φ )}}{{\sum }_{{z}^{′}}^{ } {e}^{- {E}_{q} ( z \vert x , φ )}}$$

$$where {E}_{q} ( z \vert x ) = - {g ( x )}^{T} \cdot z$$

and then using the following expression:

$$\frac{\partial KL ( q ∥ p )}{\partial φ} = ( {( g ( x ) - h - ( {J}^{T} + J ) \cdot {〈 z 〉}_{q} )}^{T} ⊙ {( {〈 z 〉}_{q} - {〈 z 〉}_{q}^{2} )}^{T} ) \cdot \frac{\partial g ( x )}{\partial φ}$$

Equation Error! Reference source not found. can therefore be simplified by dropping the dependence of p on z and then marginalizing z out of q, as follows:

$$\frac{\partial }{\partial φ} {𝔼}_{q ( ζ , z \vert x , φ )} [ log p ( x \vert ζ , z , θ ) ] \approx \frac{1}{N} {\sum }_{ρ ∼ {U ( 0 , 1 )}^{n}}^{ } \frac{\partial }{\partial ∅} log p ( x \vert ζ ( ρ ) , θ ) {\vert}_{ζ = ζ ( ρ )} \qquad ( 1 )$$

An example of a transformation from the discrete latent space to a continuous latent space is the spike-and-slab transformation:

$$r ( {ζ}_{i} \vert {z}_{i} = 0 ) = \{ \begin{matrix}∞ , & if {ζ}_{i} = 0 \\ 0 , & otherwise\end{matrix} \\ r ( {ζ}_{i} \vert {z}_{i} = 1 ) = \{ \begin{matrix}1 , & if 0 \leq {ζ}_{i} \leq 1 \\ 0 , & otherwise\end{matrix}$$

This transformation is consistent with sparse coding.

Other expansions to the continuous space are also possible. As an example, a combination of delta spike and exponential function can be used:

$$r ( {ζ}_{i} \vert {z}_{i} = 0 ) = \{ \begin{matrix}∞ , & if {ζ}_{i} = 0 \\ 0 , & otherwise\end{matrix} \\ r ( {ζ}_{i} \vert {z}_{i} = 1 ) = \{ \begin{matrix}\frac{β {e}^{β ζ}}{{e}^{β} - 1} , & if 0 \leq {ζ}_{i} \leq 1 \\ 0 , & otherwise\end{matrix}$$

Alternatively, it is possible to define a transformation from discrete to continuous variables in the approximating posterior, r(ζ|z), where the transformation is not independent of the input x. In the true posterior distribution, p(ζ|z,x)≈p(ζ|z) only if z already captures most of the information about x and p(ζ|z,x) changes little as a function of x. In a case where it may be desirable for q(ζᵢ|zᵢ,x,ϕ) to be a separate Gaussian for both values of the binary zᵢ, it is possible to use a mixture of a delta spike and a Gaussian to define a transformation from the discrete to the continuous space for which the CDF can be inverted piecewise.

### Concluding Generalities

The above described method(s), process(es), or technique(s) could be implemented by a series of processor readable instructions stored on one or more nontransitory processor-readable media. Some examples of the above described method(s), process(es), or technique(s) method are performed in part by a specialized device such as an adiabatic quantum computer or a quantum annealer or a system to program or otherwise control operation of an adiabatic quantum computer or a quantum annealer, for instance a computer that includes at least one digital processor. The above described method(s), process(es), or technique(s) may include various acts, though those of skill in the art will appreciate that in alternative examples certain acts may be omitted and/or additional acts may be added. Those of skill in the art will appreciate that the illustrated order of the acts is shown for exemplary purposes only and may change in alternative examples. Some of the exemplary acts or operations of the above described method(s), process(es), or technique(s) are performed iteratively. Some acts of the above described method(s), process(es), or technique(s) can be performed during each iteration, after a plurality of iterations, or at the end of all the iterations.

The above description of illustrated implementations, including what is described in the Abstract, is not intended to be exhaustive or to limit the implementations to the precise forms disclosed. Although specific implementations of and examples are described herein for illustrative purposes, various equivalent modifications can be made without departing from the spirit and scope of the disclosure, as will be recognized by those skilled in the relevant art. The teachings provided herein of the various implementations can be applied to other methods of quantum computation, not necessarily the exemplary methods for quantum computation generally described above.

The various implementations described above can be combined to provide further implementations. All of the commonly assigned US patent application publications, US patent applications, foreign patents, and foreign patent applications referred to in this specification and/or listed in the Application Data Sheet are incorporated herein by reference, in their entirety, including but not limited to:

U.S. Pat. No. 9,727,824; PCT application no. US2016/057208; PCT application no. US2016/047628; PCT application no. US2016/047627; PCT application no. US2017/15401; U.S. patent application No. 62/304,737; PCT application no. US2017/053303; U.S. patent application Ser. No. 15/725,600; U.S. provisional patent application No. 62/508,343; U.S. provisional patent application No. 62/598,880; U.S. provisional patent application No. 62/658,461; U.S. provisional patent application No. 62/627,579; U.S. provisional patent application No. 62/757,012; and PCT application no. US2018/065286.

These and other changes can be made to the implementations in light of the above-detailed description. In general, in the following claims, the terms used should not be construed to limit the claims to the specific implementations disclosed in the specification and the claims, but should be construed to include all possible implementations along with the full scope of equivalents to which such claims are entitled. Accordingly, the claims are not limited by the disclosure.

1. A method for machine learning over an input space comprising a plurality of input variables relating to a plurality of organisms, and at least a subset of a training dataset of samples of the respective variables, to attempt to identify the value of at least one parameter that increases the log-likelihood of the at least a subset of a training dataset with respect to a model, the model expressible as a function of the at least one parameter, the method executed by circuitry including at least one processor and comprising;
forming a latent space comprising a genetic latent subspace and an environmental subspace, each subspace comprising one or more continuous random latent variables;
forming an approximating posterior distribution over the latent space, conditioned on the input space;
forming a prior distribution over the latent space;
forming a decoding distribution over the input space, conditioned on the latent space, the decoding distribution conditioned on the random latent variables of the genetic latent subspace based on genetic covariance induced by familial relationships between organisms; and
training the model based on the encoding, prior, and decoding distributions.

2. The method of claim 1 wherein forming a decoding distribution over the input space, the decoding distribution conditioned on the random latent variables of the genetic latent subspace based on genetic covariance induced by familial relationships between organisms comprises conditioning a latent variable corresponding to an organism on a first parental latent variable corresponding to a first parent of the organism and a second parental latent variable corresponding to a second parent of the organism.

3. The method of claim 2 wherein forming a decoding distribution over the input space, the decoding distribution conditioned on the random latent variables of the genetic latent subspace based on genetic covariance induced by familial relationships between organisms further comprises conditioning the latent variable corresponding to the organism on a noise latent variable, the noise latent variable independent of the first and second parental latent variables.

4. The method of claim 1 wherein forming a decoding distribution over the input space, conditioned on the latent space comprises conditioning the decoding distribution on the random latent variables of the environmental latent subspace based on shared environmental characteristics of a subset of the plurality of organisms.

5. The method of claim 4 wherein conditioning the decoding distribution on the random latent variables of the environmental latent subspace based on shared environmental characteristics of a subset of the plurality of organisms comprises, for a first subset of family latent variables in the environmental latent subspace and a second subset of sibling latent variables, conditioning the decoding distribution on the family latent variables and sibling latent variables when generating a representation of sibling organisms and conditioning on the family latent variables exclusive of the sibling latent variables when generating a representation of a parent organism of the sibling organisms.

6. The method of claim 1 wherein training the model comprises training the model based on genetic sequence data for one or more of the plurality of organisms.

7. The method of claim 1 wherein the input space comprises diagnoses for a plurality of diseases for the plurality of organisms and training the model comprises generating one or more generated organisms each having a prediction for each of the plurality of diseases.

8. The method of claim 1 comprises predicting diagnoses for one or more of the plurality of diseases for a given organism by determining a latent representation of the given organism based on the approximating posterior distribution, sampling one or more times from the prior distribution based on the latent representation to obtain one or more samples, generating the one or more generated organisms based on the one or more samples, and determining a prediction for each disease based on the one or more generated organisms and the decoding distribution.

9. The method of claim 8 wherein forming a decoding distribution over the input space comprises conditioning the decoding distribution based on age information of the plurality of organisms and determining a prediction for each disease based on the decoding distribution comprises conditioning the decoding distribution based on an age of the given organism.

10. A machine-learning system, comprising:
at least one processor;
at least one nontransitory processor-readable medium communicatively coupled to the at least one processor, the at least one nontransitory processor-readable medium which stores at least one of processor-executable instructions or data which, when executed by the at least one processor, cause the at least one processor to attempt to identify the value of at least one parameter that increases the log-likelihood of the at least a subset of a training dataset with respect to a model, and particularly cause the processor to:
form a latent space comprising a genetic latent subspace and an environmental subspace, each subspace comprising one or more continuous random latent variables;
form an approximating posterior distribution over the latent space, conditioned on the input space;
form a prior distribution over the latent space;
form a decoding distribution over the input space, conditioned on the latent space, the decoding distribution conditioned on the random latent variables of the genetic latent subspace based on genetic covariance induced by familial relationships between organisms; and
train the model based on the encoding, prior, and decoding distributions.

11. The system of claim 10 wherein the at least one of processor-executable instructions or data which cause the at least one processor to form a decoding distribution over the input space, the decoding distribution conditioned on the random latent variables of the genetic latent subspace based on genetic covariance induced by familial relationships between organisms comprises at least one of processor-executable instructions or data which cause the at least one processor to condition a latent variable corresponding to an organism on a first parental latent variable corresponding to a first parent of the organism and a second parental latent variable corresponding to a second parent of the organism.

12. The system of claim 11 wherein the at least one of processor-executable instructions or data which cause the at least one processor to form a decoding distribution over the input space, the decoding distribution conditioned on the random latent variables of the genetic latent subspace based on genetic covariance induced by familial relationships between organisms further comprises at least one of processor-executable instructions or data which cause the at least one processor to condition the latent variable corresponding to the organism on a noise latent variable, the noise latent variable independent of the first and second parental latent variables.

13. The system of claim 10 wherein the at least one of processor-executable instructions or data which cause the at least one processor to form a decoding distribution over the input space, conditioned on the latent space comprises at least one of processor-executable instructions or data which cause the at least one processor to condition the decoding distribution on the random latent variables of the environmental latent subspace based on shared environmental characteristics of a subset of the plurality of organisms.

14. The system of claim 13 wherein the at least one of processor-executable instructions or data which cause the at least one processor to condition the decoding distribution on the random latent variables of the environmental latent subspace based on shared environmental characteristics of a subset of the plurality of organisms comprises at least one of processor-executable instructions or data which cause the at least one processor to, for a first subset of family latent variables in the environmental latent subspace and a second subset of sibling latent variables, condition the decoding distribution on the family latent variables and sibling latent variables when generating a representation of sibling organisms and conditioning on the family latent variables exclusive of the sibling latent variables when generating a representation of a parent organism of the sibling organisms.

15. The system of claim 10 wherein the at least one of processor-executable instructions or data which cause the at least one processor to train the model comprises at least one of processor-executable instructions or data which cause the at least one processor to train the model based on genetic sequence data for one or more of the plurality of organisms.

16. The system of claim 10 wherein the input space comprises diagnoses for a plurality of diseases for the plurality of organisms and the at least one of processor-executable instructions or data which cause the at least one processor to train the model comprises at least one of processor-executable instructions or data which cause the at least one processor to generate one or more generated organisms each having a prediction for each of the plurality of diseases.

17. The system of claim 10 comprises at least one of processor-executable instructions or data stored by the medium which cause the at least one processor to predict diagnoses for one or more of the plurality of diseases for a given organism by determining a latent representation of the given organism based on the approximating posterior distribution, sampling one or more times from the prior distribution based on the latent representation to obtain one or more samples, generating the one or more generated organisms based on the one or more samples, and determining a prediction for each disease based on the one or more generated organisms and the decoding distribution.

18. The system of claim 17 wherein at least one of processor-executable instructions or data which cause the at least one processor to form a decoding distribution over the input space comprises at least one of processor-executable instructions or data which cause the at least one processor to condition the decoding distribution based on age information of the plurality of organisms and determining a prediction for each disease based on the decoding distribution comprises conditioning the decoding distribution based on an age of the given organism.

19. The system of claim 10, further comprising:
at least one interface communicatively coupled to receive information from at least one quantum computing system that includes at least one quantum processor.

20. The system of claim 10, further comprising:
at least one quantum computing system that includes at least one quantum processor.
